(19) United States
(12) Patent Application Publication
(10) Pub. No.: US 2024/0009601 A1
(43) Pub. Date: Jan. 11, 2024

(54) Final Fill Assembly and Method of Integrity Testing

(71) Applicant: EMD Millipore Corporation, Burlington, MA (US)

(72) Inventors: Santosh Rahane, Harvard, MA (US); Charles Raye, Portsmouth, NH (US); An son Leong, Somerville, MA (US)

(21) Appl. No.: 18/251,558

(22) PCT Filed: Nov. 18, 2021
(86) PCT No.: PCT/US2021/059907

§ 371 Date: May 3, 2023

Related U.S. Application Data
(60) Provisional application No. 63/115,838, filed on Nov. 19, 2020.

(51) Int. Cl. B01D 35/143; G01N 15/08; A61L 2/28

(52) U.S. Cl. CPC B01D 35/143; G01N 15/0826; A61L 2/28; G01N 2015/084; A61L 2202/24; A61L 2202/21

(57) ABSTRACT

Apparatus and methods for redundant filtration assemblies containing filters comprising a multi-purpose vent port are disclosed, wherein the redundant filtration assemblies reduce the amount of components and overall size of the assemblies, promoting the minimization of product losses. A method(s) to conduct pre-use post-sterilization integrity test (PUPSIT) are also disclosed.

### RELATED APPLICATIONS

The present application claims the benefit of U.S. Provisional Patent Application No. 63/115,838, filed on Nov. 19, 2020, the entire contents of which is incorporated by reference herein in its entirety.

### FIELD OF THE DISCLOSURE

Embodiments of the present disclosure relate to the processing of biological fluids. More particularly, embodiments disclosed herein are related to integrity testing of devices used in bioprocessing.

### BACKGROUND

Single-use assemblies are increasingly being implemented throughout the manufacturing of biological products to minimize cleaning, improve efficiency and maximize flexibility as manufacturers strive to meet the demands of production schedules. Pre-sterilized single-use assemblies offer advantages to final filtration and filling operations where maintaining sterility is critical to assuring biologics and drug safety for patients. Due to the high cost of final biological products filtration, past traditional, prior art assemblies involve the use of a redundant filter in addition to a primary filter to ensure final filtration occurs without any errors. The single-use redundant filtration assemblies are referred to as SURF assemblies.

As manufacturing processes have evolved, so has the design of filter capsules. For example, past capsule filters included a traditional filter vent, which has been replaced by a specialized port that has been validated to prevent microorganisms from the outside environment from entering the aseptic flow path. This specialized port can be used for venting, sampling and for connecting an air line, thus simplifying pre-use, post sterilization, integrity testing (PUPSIT). In contrast to traditional filter vents, the aseptic multi-purpose port (otherwise known as an “AMPP”) is designed to maintain an aseptic connection while tolerating the high pressures required for filter integrity testing. In addition, pressure can be applied through the aseptic multi-purpose port following processing to recover product in the filtration system. In small volume processing or where high value drug products are being processed, this recovery step can have significant economic benefits.

Overall, the design of SURF assemblies is targeted to minimize the product losses occurring during the filtration operation and ability to recover the products in the assembly. This can be achieved by reducing the total hold-up volume of the SURF assembly or by introducing several recovery steps post filtration. Such recovery steps must not compromise the sterility of the assembly. However, past SURF assemblies have required the use of redundant final fill filters and barrier filters. Some past SURF assemblies may have included two separate filters instead of one barrier filter, whereby one filter is serves as an outlet for gas and one serves as an outlet for liquid.

A streamlined redundant filtration assembly, having fewer barrier filters and/or gas filters and/or liquid filters, wherein the hold-up volume is reduced and minimizes product losses during the filtration operation, would represent an advance in the art. A pre-use post-sterilization integrity test having fewer barrier filters and/or gas filters and/or liquid filters also represents an advance in the art.

### SUMMARY

Some embodiments described herein include a streamlined redundant filtration assembly, comprising: a main conduit for delivering a biological product, the main conduit further comprises: a primary final fill filter disposed within the main conduit; a first connector and a second connector at terminal ends of the main conduit; a clamp is disposed within the main conduit downstream of the first connector; a redundant final fill filter is disposed within the main conduit; an air line in fluid communication with the redundant final fill filter is joined to the main conduit, the air line further comprising an integrity test connection at a distal end; a vent connected to the air line; at least one vent bag is in fluid communication with the redundant final fill filter; two clamps are disposed downstream of the redundant final fill filter, wherein a pinch clamp is disposed between the two clamps; two vent bags, an air line, and optional clamps and a gas filter are in fluid communication with the primary filter; a clamp is disposed in the main conduit downstream of the primary filter; a secondary conduit is joined to the main conduit; the secondary conduit further comprises a barrier filter, the barrier filter and the secondary conduit are joined with the main conduit, a pinch clamp is disposed on the main conduit, wherein the main conduit terminates at the second connector.

In some embodiments, the redundant filtration assembly comprises an integrity test connection connected to an air supply. In some embodiments, the redundant filtration assembly comprises a gas filter downstream of the integrity test connection. In some embodiments, the redundant filtration assembly comprising two vent bags. In some embodiments, the redundant filtration assembly further comprises a sampling bag. In some embodiments, the redundant filtration assembly further comprises a clamp or a valve disposed on the air line between the two vent bags. In some embodiments, the vent is an aseptic multi-purpose port (AMPP). In some embodiments, the redundant filtration assembly further comprises a peristaltic pump having a conduit connected to the integrity test connection at a first end of the conduit. In some embodiments, the redundant filtration assembly further comprises more than one integrity test connection. In some embodiments, the redundant filtration assembly comprises a second end of the conduit connected to a different integrity test connection than the first end of the conduit. In some embodiments, the redundant filtration assembly further comprises a recirculation vessel. In some embodiments, the redundant filtration assembly further comprises a data acquisition system. In some embodiments, the redundant filtration assembly is single-use. In some embodiments, the redundant filtration assembly comprises stainless steel. In some embodiments, the redundant filtration assembly comprises stainless steel and single-use components.

Some embodiments described herein include a method of integrity testing of at least one final fill filter of the redundant filtration assembly, the method comprising: flowing a wetting liquid through the final fill filter; introducing pressurized air into the streamlined redundant filtration assembly through the air line further comprising the integrity testing connection at the distal end; draining the assembly of the wetting liquid; passing the pressurized air through the gas filter on the air inlet and through the vent and the final fill filter before exiting the streamlined redundant filtration assembly through an outlet; and performing at least one test selected from the group consisting of: a bubble point test, a diffusion test, a water flow test, and a pressure hold test. The method of claim 16, wherein the vent is an aseptic multi-purpose port (AMPP) vent port. The method of any one of claims 16 and 17, further comprising placing a clamp between the primary filter and the redundant filter, thereby avoiding fluid communication between the downstream side of the redundant filter and the air inlet for the primary filter.

In some embodiments of the method, the draining step is performed using a gravity drain. In some embodiments of the method, the draining step is performed using a blow-down. In some embodiments of the method, the final fill filter is the primary final fill filter. In some embodiments of the method, the final fill filter is the redundant final fill filter. In some embodiments, the method further comprises closing the AMPP vent port on the primary filter. In some embodiments of the method, the barrier filter is the final outlet of the pressurized air. In some embodiments, the method further comprises opening the AMPP vent port on the primary filter. In some embodiments of the method, the AMPP vent port is the final outlet of the pressurized air. In some embodiments of the method, the pressurized air passes sequentially through an air inlet for the redundant final fill filter and the redundant final fill filter and exits the redundant filtration assembly through the AMPP vent port of the redundant final fill filter. In some embodiments of the method, the pressurized air passes sequentially through an air inlet of redundant final fill filter into the redundant final fill filter and exits the redundant filtration assembly through AMPP vent port of the primary final fill filter. In some embodiments of the method, the pressurized air passes sequentially through an air inlet of redundant final fill filter into the redundant final fill filter and exits the redundant filtration assembly through an air inlet of the primary final fill filter.

Apparatus and methods for redundant filtration assemblies containing filters comprising an aseptic multi-purpose vent port (AMPP), substantially as shown in and/or described in connection with at least one of the figures, as set forth more completely in the claims, are described herein. The redundant filtration assemblies described herein reduce the number of components and overall size of the assemblies, which promotes the minimization of product losses. A method(s) to conduct pre-use post-sterilization integrity test (PUPSIT) is also developed. Various benefits, aspects, novel and inventive features of the present disclosure, as well as details of exemplary embodiments thereof, will be more fully understood from the following description and drawings.

### BRIEF DESCRIPTION OF THE FIGURES

FIG. 1 depicts an embodiment of an assembly in the prior art that uses redundant and primary filters.

FIG. 2 depicts an embodiment of the flow direction of pressurized air as it travels through the final filter and outwards from the respective barrier filter in the traditional, prior art assembly of FIG. 1.

FIG. 3 depicts some embodiments of a streamlined redundant filtration assembly that reduces the hold-up volume and minimizes product losses during filtration operations, according to some embodiments of the disclosure.

FIG. 4A and FIG. 4B depicts some embodiments of an experimental setup to compare the recovery or product losses using the redundant filtration assemblies depicted in FIG. 1 and FIG. 3.

FIG. 5 compares hold-up volumes of the streamlined assembly and the prior art assembly and shows the streamlined assembly has significantly less hold-up volume due to, at least in part, a smaller size.

FIG. 6 compares the differences between product losses for the redundant filtration assembly of FIG. 1 and the streamlined redundant filtration assembly of FIG. 3, according to some embodiments of the disclosure, after a gravity drain as recovery step is employed for both.

FIG. 7 compares the volume of unrecovered liquid as a function of recovery methods for different liquids having different viscosities.

FIG. 8A and FIG. 8B compare the impact of assembly angle on extent of product losses for the redundant filtration assembly of FIG. 1 and some embodiments of the streamlined redundant filtration assembly of FIG. 3.

FIG. 9A and FIG. 9B depicts some embodiments of the flow direction of pressurized air during the integrity testing of the primary and redundant final fill filter for the streamlined redundant filtration assembly.

FIG. 10 depicts the pressure evolution as a function of time measured using pressure sensors upstream of primary and redundant filters on the streamlined redundant filtration assembly when integrity testing the redundant final fill filter.

FIG. 11 depicts a flow direction of pressurized air during the integrity testing of the redundant final fill filter in some embodiments of the streamlined redundant filtration assembly.

FIG. 12 depicts a flow direction of pressurized air during the integrity testing of the redundant final fill filter on a variation of a streamlined redundant filtration assembly.

FIG. 13 depicts a pressure evolution as a function of time measured using pressure sensors upstream of primary and redundant filters in some embodiments of the streamlined redundant filtration assembly.

FIG. 14 depicts a flow direction of pressurized air during the integrity testing of the redundant final fill filter on a variation of a redundant filtration assembly.

FIG. 15 depicts a flow direction of pressurized air during the integrity testing of the redundant final fill filter on some embodiments of a streamlined redundant filtration assembly.

FIG. 16 depicts a flow direction of pressurized air during the integrity testing of the redundant final fill filter on some embodiments of a streamlined redundant filtration assembly.

FIG. 17 depicts a flow direction of pressurized air during the integrity testing of the redundant final fill filter on a variation of a streamlined redundant filtration assembly, according to some embodiments of the disclosure.

FIG. 18 depicts a flow direction of pressurized air during the integrity testing of the redundant final fill filter for some embodiments of a streamlined redundant filtration assembly.

FIG. 19 depicts a flow direction of pressurized air during the integrity testing of the redundant final fill filter for some embodiments of a streamlined redundant filtration assembly.

### DETAILED DESCRIPTION OF SOME EMBODIMENTS

The manner in which the features disclosed herein can be understood in detail, a more particular description of the embodiments of the disclosure, briefly summarized above, may be had by reference to the appended drawings. It is to be noted, however, that the appended drawings illustrate only some embodiments of this disclosure and are therefore not to be considered limiting of its scope, for the embodiments described and shown may admit to other equally effective embodiments. It is also to be understood that elements and features of one embodiment may be found in other embodiments without further recitation and that identical reference numerals are sometimes used to indicate comparable elements that are common to the figures.

### Definitions

The term “barrier filter,” as used herein, has both hydrophobic and hydrophilic components, and hence can be used in place of two filters, whereby one is hydrophilic and another being hydrophobic for gas.

The term “depth filter,” as used herein, is a filter that achieves filtration within the depth of the filter material. Particle separation in depth filters results from entrapment by or adsorption to, the fiber and filter aid matrix comprising the filter material.

The terms “sterile” and “sterilized,” as used herein, are defined as a condition of being free from contaminants and, particularly within the bioprocessing industry, free from pathogens, such as undesirable viruses, bacteria, germs, and other microorganisms. Relatedly, the terms “bioburden-reduced” and “bioburden reduction” (e.g., by a non-sterilizing dose of gamma or X-ray radiation <25 kGy) may be substituted for certain embodiments that do not necessitate a sterile claim.

The term “upstream,” as used herein, is defined as first step processes in the processing of biological materials, such as microbes/cells, mAbs, ADCs, proteins, including therapeutic proteins, viral vectors, etc., are grown or inoculated in bioreactors within cell culture media, under controlled conditions, to manufacture certain types of biological products.

The term “downstream,” as used herein, indicates those processes in which biological products are harvested, tested, purified, concentrated and packaged following growth and proliferation within a bioreactor.

The term “clarification,” as used herein, is defined as a downstream process, wherein whole cells, cellular debris, soluble impurities (HCP and/or DNA), suspended particles, and/or turbidity are reduced and/or removed from a cell culture feedstream using centrifugation and/or depth filtration. The terms “clarify,” “clarification,” “clarification step,” and “harvest” generally refer to one or more steps used initially in the purification of biomolecules. The clarification step generally comprises the removal of whole cells and/or cellular debris during a harvest operation from a bioreactor but may also comprise turbidity reduction for downstream process intermediates or pre-filters to protect other sensitive filtration steps, e.g. virus filtration.

The term “purification” is defined as a downstream process, wherein bulk contaminants and impurities, including host cell proteins, DNA and process residuals are removed from the product stream.

The term “polishing” is defined as a downstream process, wherein trace contaminants or impurities that resemble the product closely in physical and chemical properties are eliminated from the purified product stream.

The term “impurity” or “contaminant” as used herein, refers to any foreign or disfavored molecule, including a biological macromolecule such as DNA. RNA, one or more host cell proteins, endotoxins, lipids, flocculation polymer, surfactant, antifoam additive(s), and one or more additives which may be present in a sample containing the target molecule that is being separated from one or more of the foreign or disfavored molecules using a process described herein. Additionally, such impurity may include any reagent which is used in a step which may occur prior to the method of the invention. Impurities may be soluble or insoluble.

The term “hold-up volume” as used herein, refers to the volume of the mobile phase within the redundant filtration assembly during use.

Assembly

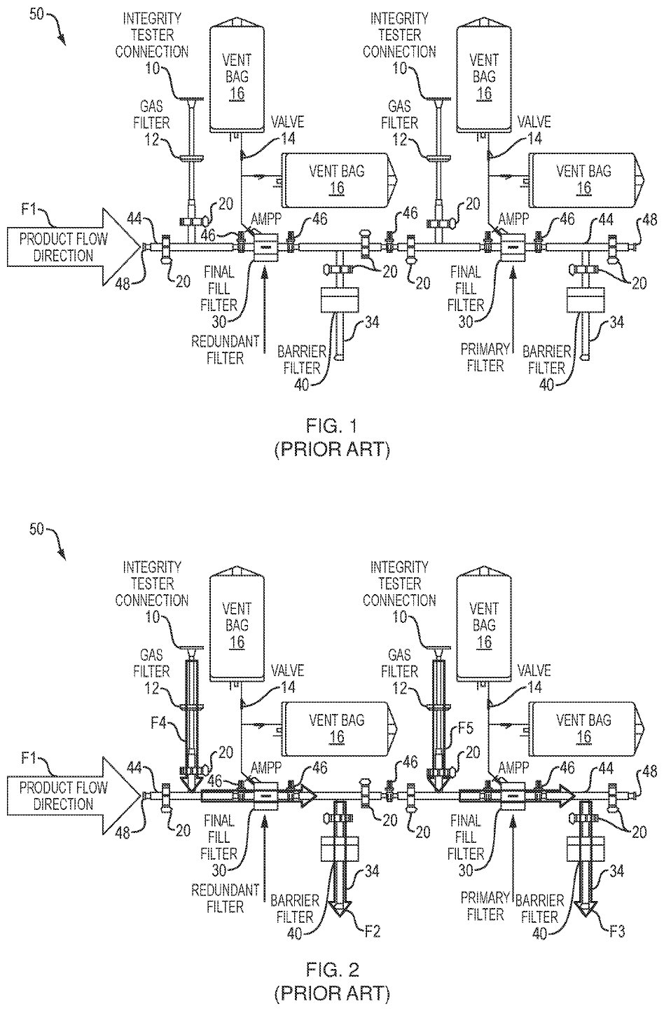

Turning to the figures, FIG. 1 depicts a typical prior art redundant filtration assembly that uses redundant and primary filters, where the filter maybe a Millipak® Final Fill filter or any other sterile filter. With most other sterile filters, air line for integrity testing cannot be directly connected to the vent of the filter. Therefore, as shown in FIG. 1, an additional inlet for the integrity testing is needed. In addition, prior to integrity testing, the filter must be wetted using wetting fluid at specific pressure and flow rates. Typically, this wetting fluid must pass through the respective filter and exit the assembly using another connection. This connection can contain any sterile filter or a pre-sterilized bag, or a hydrophilic/phobic filter (shown in FIG. 1). Similarly, the pressurized air used for integrity testing must also pass through the respective filter and exit the assembly using a connection. This connection can contain a gas filter or a hydrophilic/phobic filter (shown in FIG. 1). In summary, the connection for the liquid and gas to exit the assembly after passing through the respective filter may contain a hydrophilic/phobic filter or separate gas and liquid filters. For the example schematic shown in FIG. 1, during the wetting process, the wetting fluid exits the assembly through the Millipak® Barrier filter downstream of the respective Final Fill filter, whereby the Millipak® Barrier filter is a hydrophilic/hydrophobic filter.

In addition to the barrier filter, the assembly might contain several vent bags to ensure proper venting of the assembly during the wetting process or prior to the filtration step. These vent bags are pre-sterilized and are connected to the vent port of the final fill filter. There may be an additional hydrophobic, gas filter on the air line for integrity testing to ensure that the pressurized air introduced into the redundant filtration assembly is sterile and does not compromise the sterility of the assembly during the operation. There may be additional pressure sensors upstream of each of the final fill filters to track the pressure during different steps of the final fill operation.

FIG. 2 depicts the flow direction of the pressurized air as it travels through the final filter of the traditional final fill assembly and outwards from the respective barrier filter in prior art methods for integrity testing. Each of the final fill filters has an inlet connection connected to the source of air. The barrier filter can be replaced with any appropriate gas filter. The pre-use integrity testing of the two filters on the assembly as a part of PUPSIT operation is generally done one at a time. For example, the primary filter is integrity tested first with the redundant filter portion of the assembly clamped off. For example, this clamp can be placed between the connections for barrier filter downstream of the redundant final fill filter and connection for the air inlet for integrity testing of the primary filter. After the integrity tests are complete, this clamp can be removed for the final filtration operation. Flow directions F1, F2, F3, F4, and F5, are shown.

Streamlined Redundant Filtration Final Fill Assembly Design

Some embodiments of the disclosure describe a streamlined redundant filtration assembly that minimizes the hold-up volume for the product thereby minimizing the potential product loss, and also a method of integrity testing the filters on the assembly. Some embodiments of the assembly include two or more filters, i.e., redundant. Accordingly, some embodiments of the redundant filtration assembly comprise two final fill filters at minimum. There can be fewer barrier filters as shown by the streamlined redundant filtration assembly in the FIG.s.

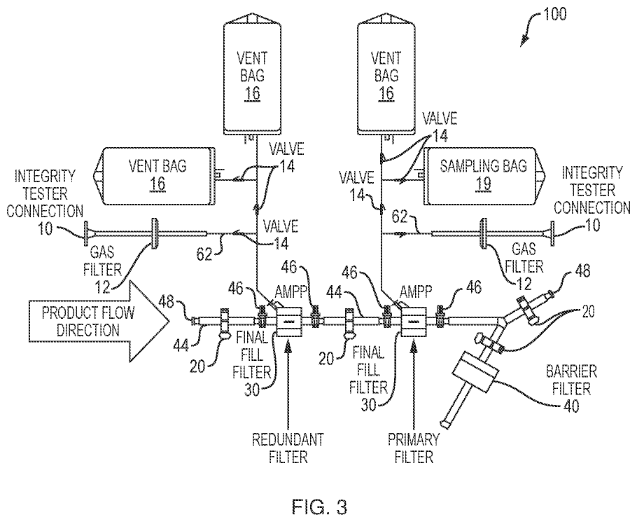

FIG. 3 depicts some embodiments of a streamlined redundant filtration assembly that reduces the hold-up volume and minimizes product losses during filtration operations. The assembly shown in the FIG. 3 consists of fewer total parts as compared to the assembly shown in FIG. 1. FIG. 3 depicts a streamlined redundant filtration assembly 100. The streamlined redundant filtration assembly 100 comprises a main conduit 44, through which a product, i.e., a biological product, flows. The main conduit 44 comprises a first connector 48 and a second connector 48 at terminal ends on the main conduit 44. A pinch clamp 20 is disposed within the main conduit 44 downstream of the first connector 48. A redundant final fill filter 30, such as a Millipak® Final Fill filter, marketed by EMD Millipore Corporation, Burlington, MA, USA, is disposed within the main conduit 44. An air line 62 is in fluid communication with the redundant final fill filter 30. The air line 62 comprises an integrity test connector 10 at a distal end, which may be connected to an air supply for integrity testing. A gas filter 12 is optionally provided downstream of the integrity test connector 10. After the integrity test connector 10, two vent bags 16 are in fluid communication with the redundant final fill filter 30. A clamp or valve 14 is optionally disposed on the air line 62 between the vent bags 16. Two clamps 46, such as tri-clamps, to connect sanitary fittings, are disposed downstream of the redundant final fill filter 30, wherein a pinch clamp is disposed between the two clamps 46. A primary filter 30, such as a final fill filter 30, is disposed within the main conduit 44. Two vent bags 16, or a vent bag 16 and a sampling bag 19, an air line 62, and optional clamps 14 and gas filter 12 are in fluid communication with the primary filter 30 similarly as described above. A clamp 46 is disposed in the main conduit 44 downstream of the primary filter 30. A secondary conduit 34 joins the main conduit 44. The secondary conduit 34 comprises a barrier filter 40, such as a Millipak® Barrier filter. After the barrier filter 40, the secondary conduit 34 joins the main conduit 44. A pinch clamp is then disposed on the main conduit 44, which terminates at the second connector 48. In certain embodiments, the tri-clamp 46 and respective sanitary fitting may be replaced using a hose-barb fitting in combination with a suitable hose clamp.

At first, the air line required to perform the integrity testing is connected to the vent, which is referred to as aseptic multi-purpose port (AMPP), instead of a dedicated connection for air lines. This reduces the need for several tubings and connections. In addition, the barrier filter downstream of the redundant filter has been removed as compared to the assembly in FIG. 1. In other embodiments, a combination of gas and liquid filter used instead of barrier filter can also be removed to streamline a redundant filtration assembly requiring a combination of a gas and liquid filter instead of barrier filter only downstream of the primary final fill filter. As a result of these changes, the redundant filtration assembly shown in FIG. 3 is smaller and contains fewer connections as compared to the redundant filtration assembly shown in FIG. 1. Fewer connections also result in a lessened risk of sterility compromise through the connections.

The two assemblies shown FIG. 1 and FIG. 3 were compared to each other by performing recovery analysis. Each assembly was tested with three solutions of different viscosities: water and solutions of 15% and 18% polyethylene glycol (PEG) with viscosities of approximately 25 and 50 centipoise (cP), respectively, to simulate different drug products. For water and the higher viscosity solutions, volumes were corrected for solution density. Studies were performed with the main flow-path in both a horizontal position and at a 45-degree angle. In addition, unrecoverable product from the streamlined redundant filtration assembly shown in FIG. 3 was also determined with the flow-path at angles of 65 and 90 degrees. The recovery analysis is compared for different methods of recovery. The recovery methods include no recovery, gravity draining and blow-down at different pressures.

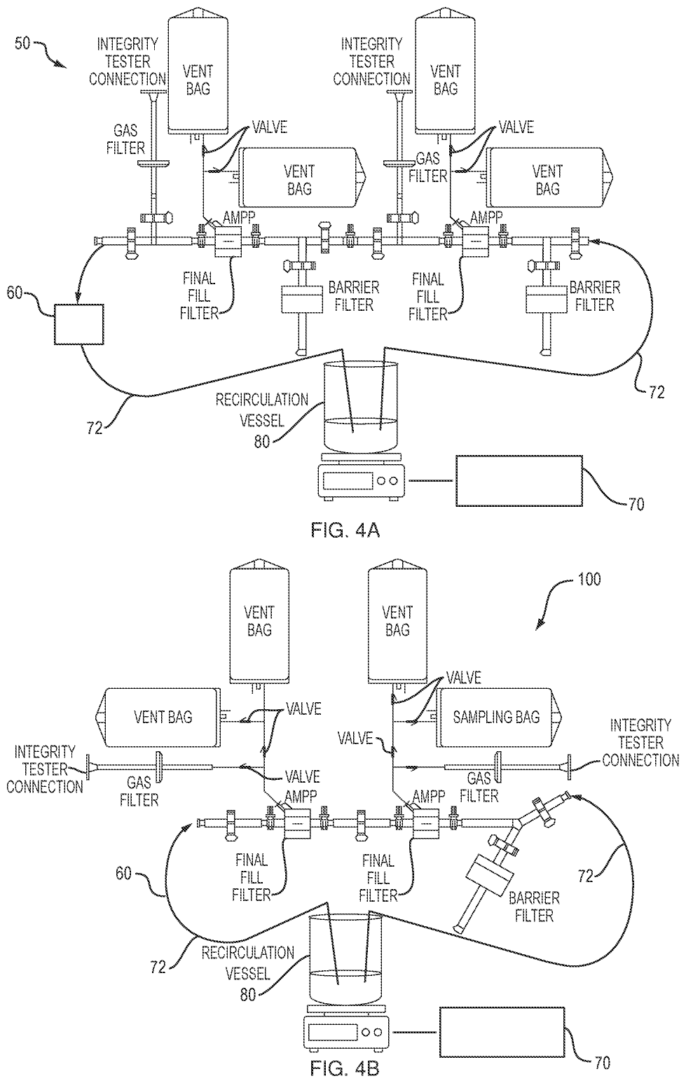

FIG. 4 depicts an experimental setup to compare the recovery or product losses using the redundant filtration assemblies depicted in FIG. 1 and FIG. 3. The setup contains a recirculation vessel and a data acquisition system to measure the mass (and volume) of the product lost after a certain recovery step. A peristatic pump is used to circulate the liquid through the assembly from the circulation tank. Before testing, the empty recirculation vessel and vessel filled with test fluid were weighed. To measure the volume of liquid held in the system, the assembly was wet with test fluid to simulate standard processing conditions. The inlet, outlets and lines to vent bags were open before introducing liquid, and lines to sampling bags, barrier filters and air lines were closed with clamps. Fluid was pushed through the assembly using the peristaltic pump at ˜2.7 mL/min (10 psi) for water and ˜200 mL/min (30 psi) for the PEG solutions. Air was vented from the filters and collected in vent bags. After venting, all vents were closed. The difference in weight of the recirculation vessel before and after assembly wetting was used to calculate the unrecovered liquid or hold-up volume of the assemblies. FIG. 4A shows the experimental setup for the redundant filtration assembly of FIG. 1. FIG. 4B shows the experimental setup for some embodiments of the streamlined redundant filtration assembly 100 of FIG. 3. Both the experimental setups of FIGS. 4A and 4B comprise a peristaltic pump 60 having a conduit 72 connected to an integrity test connector 48 and a recirculation vessel 80 and a data acquisition system such as a balance. A second end of a conduit 72 is connected to a second integrity test connector 48 after traveling through a media within the fluid in the recirculation vessel 80.

As shown in FIG. 5, the streamlined assembly according to embodiments of the disclosure has significantly less hold-up volumes due to, at least in part, smaller size. When no recovery is attempted, about 325 mL of product maybe lost with the traditional assembly as compared to approximately 270 mL or lower with the streamlined redundant filtration assembly. Due to the high value of the product at this step, this can account large amount of savings for the process.

After analyzing the hold volume, clamps on the outlet and air lines were opened on both traditional and streamlined assemblies; in the streamlined assembly, the AMPP was also opened. Assemblies were drained for 20 minutes into the recirculation vessel. The difference of the volume of circulation vessel after wetting and the gravity drain was calculated to obtain the recovery using gravity drain step.

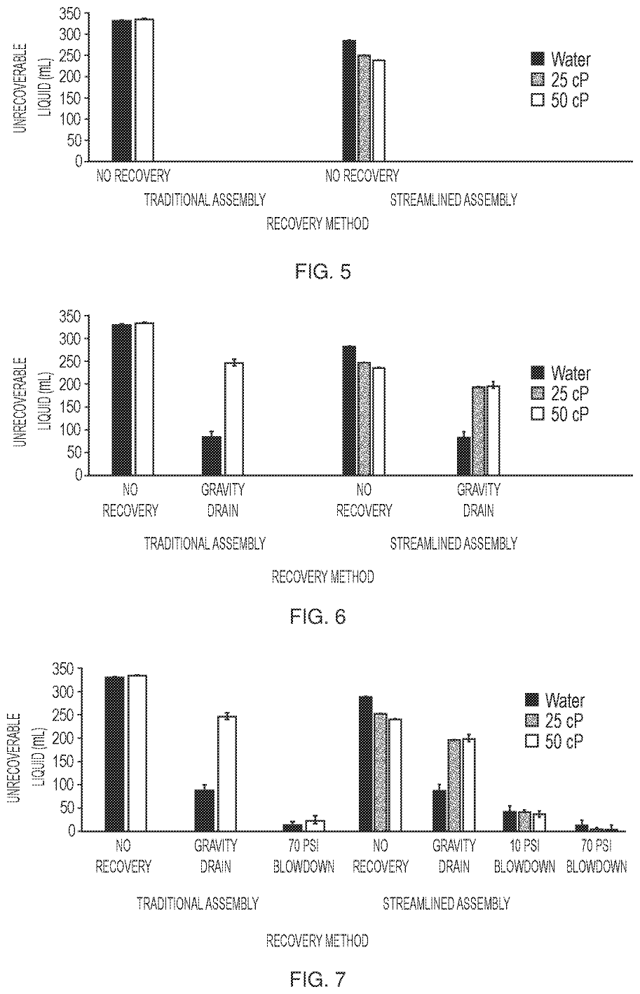

FIG. 6 depicts the difference between product losses for the redundant filtration assembly of FIG. 1 and the streamlined redundant filtration assembly of FIG. 3, according to some embodiments of the disclosure, after a gravity drain as recovery step is employed for both. FIG. 6 shows that the unrecovered liquid or product loss is similar for the two redundant filtration assemblies when gravity drain is performed as a recovery step and water is used as the liquid, the streamlined assembly shows lower product losses for viscous liquids. This improvement is due to lower hold-up volume of the streamlined assembly and is a direct result of the novel design.

After gravity draining the assembly, the rest of the liquid held in the assembly is recovered by blowing down with the help of pressurized air. Because air source is connected to the assembly at two different locations for the two assemblies, the protocol for the blow down was slightly different in each case. For the traditional, prior art redundant filtration assembly, blow-down at 70 PSI (pounds per square inch) was performed through the filter's inlet. The main flow-path upstream of the secondary filter was closed and the air source to that filter was connected to the air line. The air-line was opened, the secondary filter was pressurized to 70 PSI and drained liquid was collected. The air source was moved to the primary filter air line, the secondary filter was isolated by clamping between the two filters, and the primary filter was blown down.

For the streamlined redundant filtration assembly, blow-down was performed sequentially at 10 PSI and then 70 PSI through the AMPP. The tubing connecting the vent and sample bags to the air line was closed with valves. The air source was connected to the secondary filter through the AMPP, and the AMPP on the primary filter was closed. The air line was opened, pressurizing the secondary filter to 10 psi and drained liquid was collected. The air source was moved to the primary filter air line, connected through the AMPP, the secondary filter was isolated by clamping between the two filters and the primary filter was blown down at 10 PSI. After the 10 PSI test, the procedure was repeated with pressurized air at 70 PSI.

FIG. 7 depicts unrecovered liquid as a function of recovery methods for different liquids having varied viscosities to simulate drug product. FIG. 7 shows that using blowdown, the product losses can be minimized to almost very small amounts compared to the hold-up volume. However, when blow-down is attempted, it may create air-water interface with the drug product being filtered. This air-water interface may create a large amount of forming which may be detrimental to the product quality. Therefore, while blow-down procedure can minimize the product losses, product quality considerations are also important.

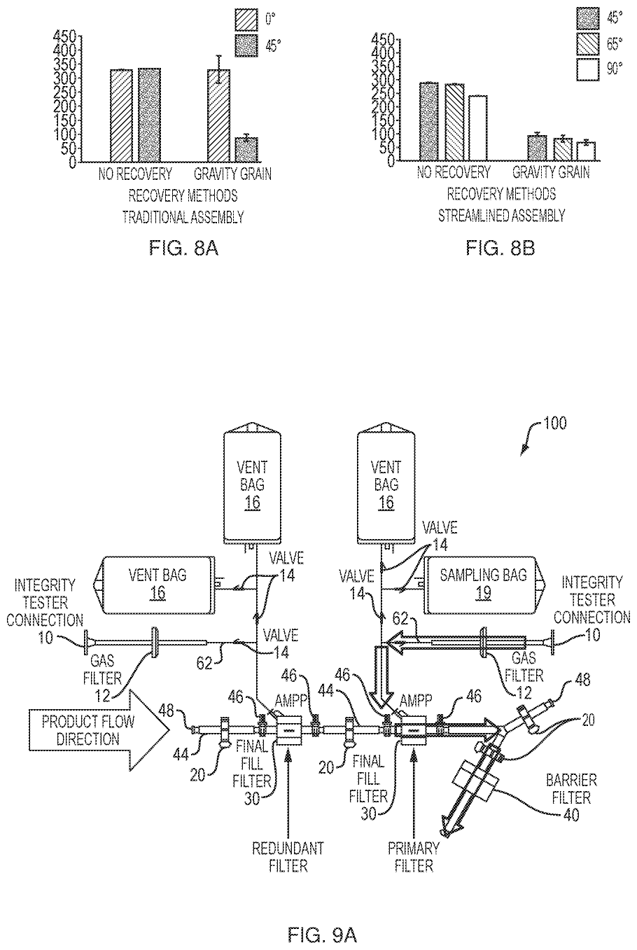

FIG. 8 depicts the impact of assembly angle on extent of product losses. Recovering liquid from the assembly using gravity is only possible if the main axis of the product flow-path is at an angle with redundant filter at a higher level compared to the primary filter rather than in the horizontal position. This modification to assembly orientation means at least 70% of liquid in the assemblies can be recovered using gravity with no additional recovery steps. Increasing the angle of the main flow-path in the streamlined assembly from 45 to 65 or 90 degrees resulted in slightly higher volume recovery, which may be worth considering for high value products. However, when the system is at 90 degrees, venting the filters became more difficult, reflected by the presence of more air and lower volume of liquid in the system.

Integrity testing was performed using an automated integrity tester as are known to those in the art. At least one such integrity tester is Integritest® 5 integrity tester, as marketed by EMD Millipore Corporation. Integritest® 5 integrity tester supports traditional tests, such as diffusion, bubble point, HydroCORR™, and pressure hold tests. Bubble point tests use the tangent method, taking pressure decay measurements at different applied pressures to map the filter's integrity profile.

The pass/fail of the integrity test is determined based on measurement of the bubble point of the filter. Bubble point is defined as the pressure at which a bulk gas flow is observed through the filter. A bubble point result higher than the specified bubble point is considered a passed integrity test and a lower than the specified bubble point is defined as a failed integrity test. The automated integrity tester relies on the ideal gas flow principles (PV=nRT, where P is pressure, V=volume, n=number of molecules, R=gas constant and T=temperature). Typically, pressure is applied onto the filter and gas flow is measured. Prior to the bulk gas flow, the flow through the wetted filter increases linearly with the increase in pressure. This is referred to as diffusive gas flow. Beyond pressure higher than the bubble point, flow rate increasing exponentially with the increase in pressure as the gas can flow through the filter pores. The point of intersection between these two curves is referred to as the bubble point.

Automated integrity testers have some limitations on determining bubble points. For example, the tester may show an “Invalid” result in the case wherein it takes too long time to obtain the bulk flow or takes too short a time to obtain the bulk flow. For example, the Millipak® Final Fill filters have a specified bubble point of 50 PSI. When testing with automated integrity tester, the tester will automatically pressurize the filter up to 80% of the specified bubble point and start measuring the gas flow. Once this pressure is stabilized, the pressure is automatically increased by 1-2 PSI each iteration until a bulk gas flow is achieved through the filter.

As discussed previously, the traditional, prior art redundant filtration assembly can be tested for integrity as shown in FIG. 2, where each of the filters on the assembly is integrity tested separately with a barrier filter (or a similar gas filter) used as an outlet for the pressurized air used for testing.

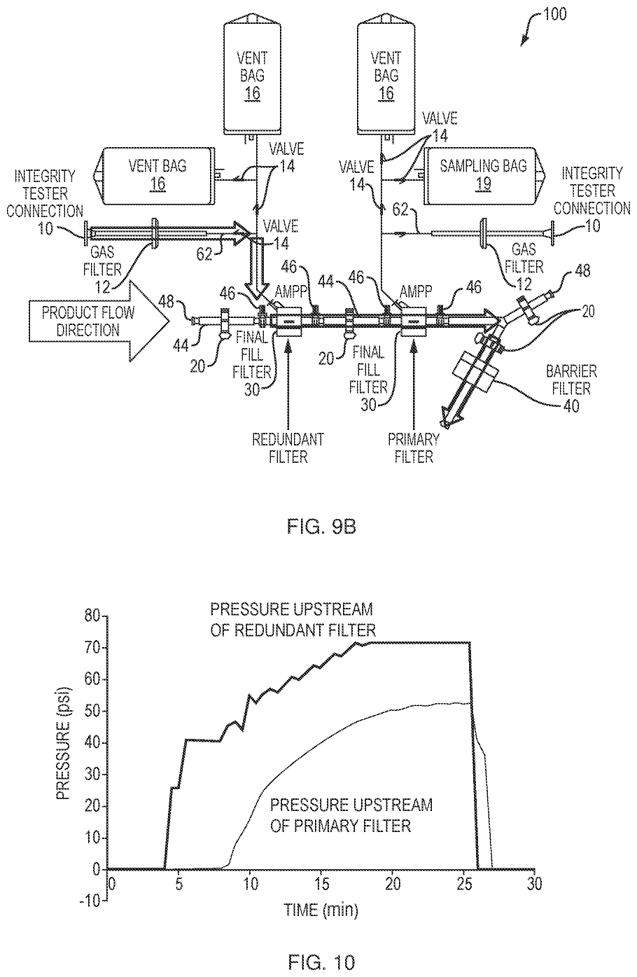

FIG. 9A and FIG. 9B depict some embodiments of the flow direction of pressurized air during the integrity testing of the primary final fill filter of a streamlined redundant filtration assembly. To conduct the integrity testing on the filters on the streamlined assembly, such as the redundant filtration assembly 100, first the filters are wet by flowing the wetting liquid through both the filters of the assembly. After wetting, the primary filter is integrity tested first. For integrity testing, the pressurized air is introduced into the assembly as shown by the arrows in FIG. 9. Prior to integrity testing, a clamp is placed between the primary and redundant filter to avoid air flow to the downstream side of the redundant filter from the air inlet for the primary filter. In addition, the assembly is gravity drained. In case the assembly is horizontal, and the gravity drain is not efficient, a blow-down at a very low pressure (that is significantly lower than bubble point) can be performed to drain the liquid in the assembly. Due to the position of the clamp and availability of the barrier filter on the downstream side of primary filter, during the integrity testing, the pressurized air passes through the gas filter on the air inlet, and through the primary filter via the AMPP vent port and exits the assembly through the barrier filter. FIG. 9A depicts some embodiments of a flow direction through the primary filter 30 of the streamlined redundant filtration assembly 100. Flow of the pressurized air through the final fill filter 30, a second final fill filter 30 and the barrier filter 40 is depicted in FIG. 9A. FIG. 9B depicts some embodiments of a flow direction through the redundant filter 30 of the redundant filtration streamlined assembly 100. Flow through the final fill filter 30 and the barrier filter 40 is depicted in FIG. 9B.

As shown in Table 1, all the tests showed that the bubble point was observed to be higher than the specified bubble point. Therefore, all the tests showed the integrity test was passed.

TABLE 1
Integrity testing of primary filter on streamline assembly
using Integritest ® 5 integrity tester

| | Specified Bubble | Measured Bubble | |
|---|---|---|---|
| Filter on Assembly | Point (psi) | Point (psi) | Test Result |
| Primary Filter | 50.0 | 53.7 | Pass |
| Primary Filter | 50.0 | 56.2 | Pass |
| Primary Filter | 50.0 | 56.3 | Pass |
| Primary Filter | 50.0 | 56.0 | Pass |
| Primary Filter | 50.0 | 56.3 | Pass |

Integrity Testing of the Redundant Filter on the Streamlined Redundant Filtration Assembly Using Barrier Filter as the Outlet for Pressurized Air

When compared to the traditional assembly, the streamlined redundant filtration assembly does not contain a barrier filter downstream of the redundant filter. Therefore, there is no direct outlet for the pressurized air. As a result, a different outlet must be chosen for the pressurized air during integrity testing. FIG. 9A and FIG. 9B shows flow direction of pressurized air during integrity testing of the integrity testing on redundant filter, whereby the barrier filter downstream filter is used as a final outlet for the air. Prior to integrity testing, the clamp placed between the primary and redundant filter is removed and the AMPP vent port on the primary filter is closed. In addition, the rest of the assembly is gravity drained. In case the assembly is horizontal, and the gravity drain is not efficient, a blow-down at very low pressure (that is significantly lower than bubble point) can be performed to drain the liquid in the assembly. As a result of this setup, the pressurized air travels through the inlet for the redundant filter, followed by the redundant filter via the AMPP vent port. The air exits the redundant filter and travels through the primary filter and barrier filter before exiting the assembly.

Table 2 shows the result of integrity test for redundant filter when the travel direction for pressurized air is as shown in FIG. 9. The automated tester could not obtain a result due to the limitation. In such cases, it is worth understanding the pressure evolution upstream of both primary and redundant filters.

FIG. 10 depicts the pressure evolution as a function of time measured using pressure sensors upstream of primary and redundant filters on the redundant filtration assembly.

TABLE 2
Integrity testing of redundant filter on streamlined assembly
using Integritest ® 5 integrity tester

| | Specified Bubble | Measured Bubble | |
|---|---|---|---|
| Filter on Assembly | Point (psi) | Point (psi) | Test Result |
| Redundant Filter | 50.0 | No measurement | Invalid |

FIG. 10 shows the pressure traces upstream of both the filters on assembly. As shown, the automated tester fails to identify a bubble point for the redundant filter ever past the specified bubble point. As shown, the bubble point was not measured even at the pressure of 70 PSI upstream of redundant filter (blue trace). This results from the primary filter acting as another restriction for the pressurized air and the pressure between the redundant filter and primary filter continues to rise even beyond an expected bubble point for the redundant filter (50 PSI). This result is unexpected and shows the inability to perform integrity test with the travel direction for air as shown in FIG. 9A and FIG. 9B.

Integrity Testing of the Redundant Filter on the Streamlined Assembly Using Vent on the Primary Filter as an Outlet.

When compared to the traditional, prior art assembly, the streamlined redundant filtration assembly does not contain a barrier filter downstream of the redundant filter. Therefore, there is no direct outlet for the pressurized air. As a result, a different outlet must be chosen for the pressurized air during integrity testing. As shown in Table 2 and FIG. 10, using the barrier filter downstream of the primary filter does not result in successful test.

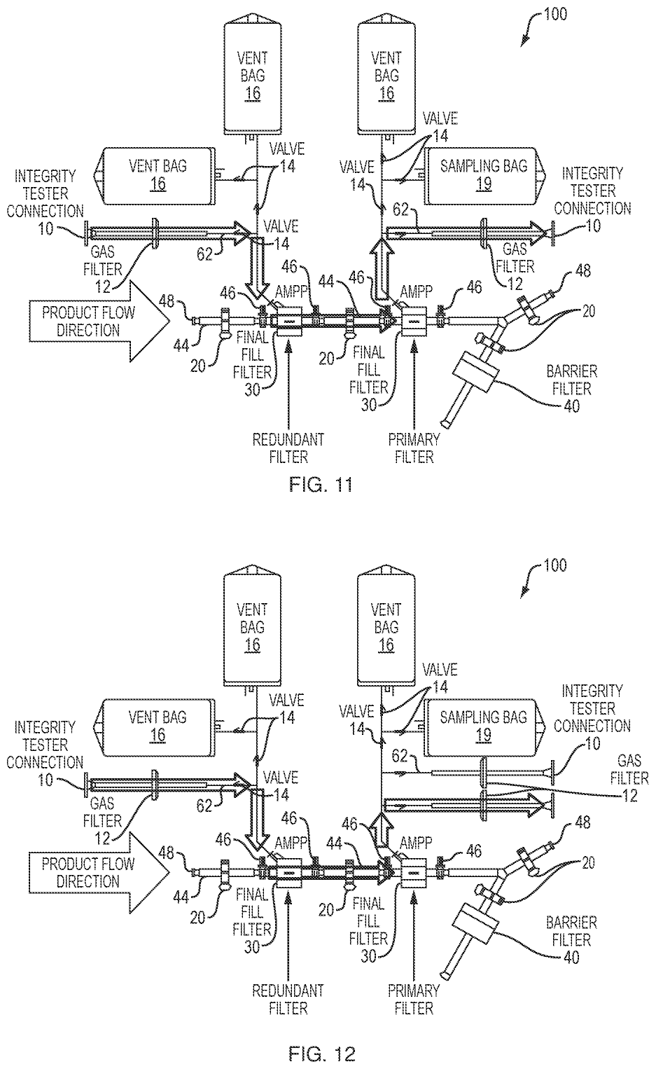

FIG. 11 depicts a flow direction of pressurized air during the integrity testing of the redundant final fill filter on the streamlined redundant filtration assembly. FIG. 11 shows a flow direction of pressurized air during integrity testing of a redundant filter, whereby the AMPP vent port on primary filter is used as a final outlet for the air. After integrity testing the primary filter, the clamp placed between the primary and redundant filter is removed. In addition, the rest of the assembly is gravity drained through the primary filter. In case the assembly is horizontal, and the gravity drain is not efficient, a blow-down at very low pressure (that is significantly lower than bubble point) can be performed to drain the liquid in the assembly. After draining any wetting liquid from the assembly, the AMPP vent port on the primary filter is opened. As a result of this setup, the pressurized air primarily travels through the inlet for the redundant filter, followed by the redundant filter via the AMPP vent port. The air exits the redundant filter and travels through AMPP vent port on primary filter before exiting the assembly.

TABLE 3
Integrity testing of redundant filter on streamlined assembly
using Integritest ® 5 integrity tester

| | Specified Bubble | Measured Bubble | |
|---|---|---|---|
| Filter on Assembly | Point (psi) | Point (psi) | Test Result |
| Redundant Filter | 50.0 | 55.2 | Pass |
| Redundant Filter | 50.0 | 54.2 | Pass |
| Redundant Filter | 50.0 | 56.0 | Pass |

Table 3 shows the result of integrity tests for redundant filter when the travel direction for pressurized air is as shown in FIG. 11. The automated tester showed results as expected with bubble point measurements higher than the specified bubble point of 50 PSI and passed the integrity test.

FIG. 12 shows some embodiments of a flow direction of pressurized air during integrity testing of a redundant filter, whereby the AMPP vent port on primary filter is used as a final outlet for the air. However, in comparison to the assembly shown in FIG. 11, the assembly of FIG. 12 contains an additional port and gas filter for the air to exit the assembly.

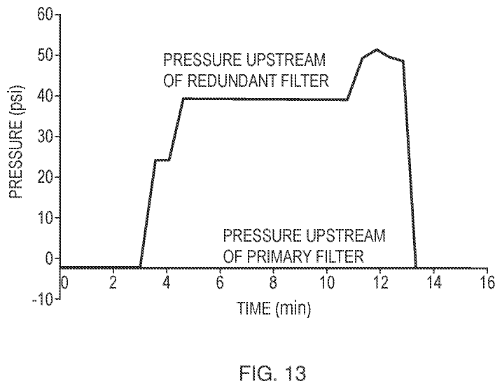

FIG. 13 depicts a pressure evolution as a function of time measured using pressure sensors upstream of primary and redundant filters on some embodiments of the streamlined redundant filtration assembly. FIG. 13 shows the pressure traces upstream of both the filters on assembly. Because the pressurized air is able to exit the AMPP vent port of the primary filter on the assembly, the primary filter does not create restriction for the air and integrity test is successfully completed. As expected, the bubble point of higher than 50 PSI was measured resulting in the test to pass. As shown by the pressure traces, the pressure upstream of the primary filter maintains around 0 PSI and pressure upstream of the redundant filter never exceeds significantly higher than the bubble point as shown in FIG. 10. Unexpectedly, this method of integrity test works despite of the absence of the barrier filter or gas filter downstream of the redundant filter. The method can be used with different embodiments of traditional assemblies as well.

### Integrity Testing of the Redundant Filter on the Traditional Assembly Using the Integrity Tester Connection as an Inlet and the Integrity Tester Connection of the Primary Filter as an Outlet.

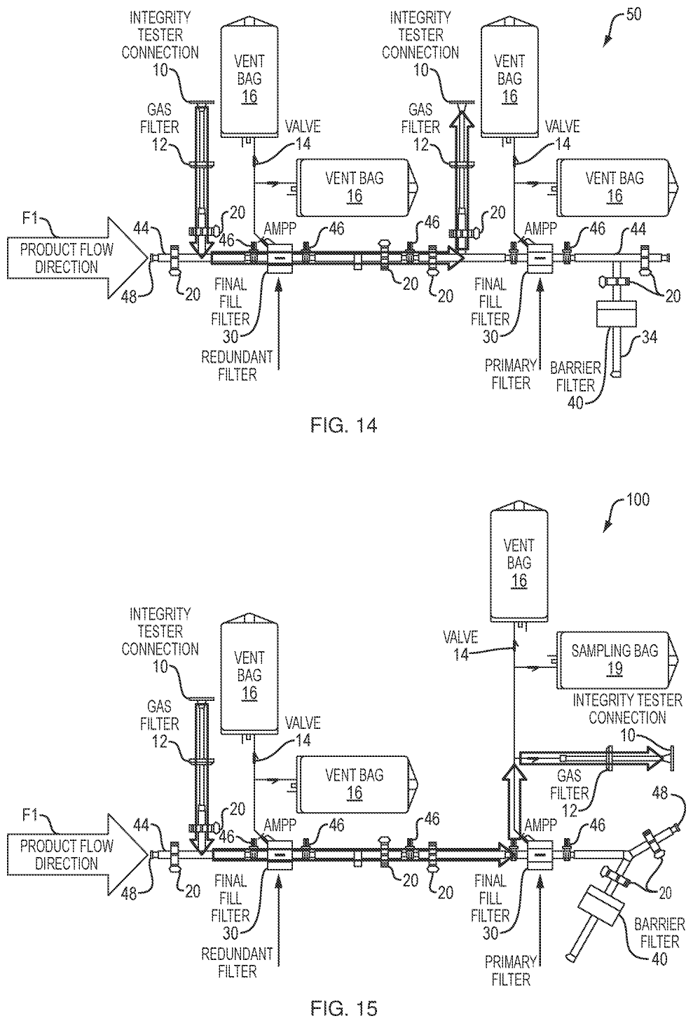

FIG. 14 depicts some embodiments of a flow direction of pressurized air during the integrity testing of the redundant final fill filter on a variation of a traditional redundant filtration assembly. FIG. 14 shows a flow direction of pressurized air during integrity testing of the redundant filter, whereby the air inlet of the primary filter is used as a final outlet for the air. As a result of this configuration, the pressurized air travels through the inlet for the redundant filter, followed by the redundant filter via the AMPP vent port. The air exits the redundant filter and travels through the inlet for the air for primary filter. This flow path may enable removing the barrier filter downstream of the redundant filter.

Integrity Testing of the Redundant Filter on the Streamlined Assembly Using the Integrity Tester Connection of the Redundant Filter as an Inlet and the Integrity Tester Connection of the Primary Filter as an Outlet.

FIG. 15 depicts a flow direction of pressurized air during the integrity testing of the redundant final fill filter on a streamlined redundant filtration assembly. FIG. 15 shows a flow direction of pressurized air during integrity testing of the redundant filter, whereby the air travels from the integrity tester connection for the redundant filter through the gas filter and through the redundant filter to exit the integrity tester connection for the primary filter. As a result of this configuration, the pressurized air travels through the inlet for the redundant filter, followed by the redundant filter via the AMPP vent port. The air exits the redundant filter and travels through the inlet for the integrity tester connection for the primary filter via the AMPP vent port of the primary filter.

### Integrity Testing of the Redundant Filter on the Streamlined Assembly Using the Integrity Tester Connection of the Redundant Filter as an Inlet and an Additional Gas Filter Connection to the AMPP of Primary Filter as an Outlet.

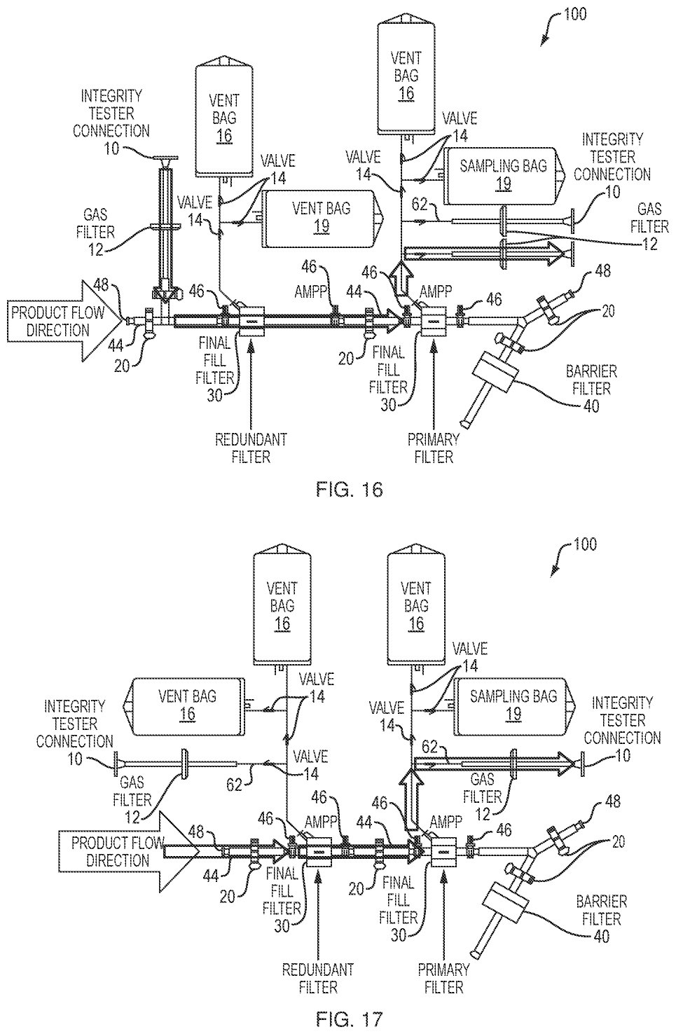

FIG. 16 depicts a flow direction of pressurized air during the integrity testing of the redundant final fill filter on a streamlined redundant filtration assembly. FIG. 16 shows a flow direction of pressurized air during integrity testing of the redundant filter, whereby the air travels from the integrity tester connection for the redundant filter through the gas filter and through the redundant filter to exit an additional gas filter connected to the primary filter through its AMPP. As a result of this configuration, the pressurized air travels through the inlet for the redundant filter, followed by the redundant filter via the AMPP vent port. The air exits the redundant filter and travels through the additional gas filter provided via the AMPP vent port of the primary filter.

### Integrity Testing of the Redundant Filter on the Streamlined Assembly Using Product Inlet for Air-Source and Vent on the Primary Filter as an Outlet

FIG. 17 shows some embodiments for a method of testing the redundant filter whereby the air enters through the inlet of the filter, travels through the filter and exits the vent on the primary filter. FIG. 17 depicts a flow direction of pressurized air during the integrity testing of the redundant final fill filter on some embodiments of a streamlined redundant filtration assembly.

### Integrity Testing of the Redundant Filter on the Streamlined Assembly Using Product Inlet for Air-Source and Vent on the Primary Filter as an Outlet

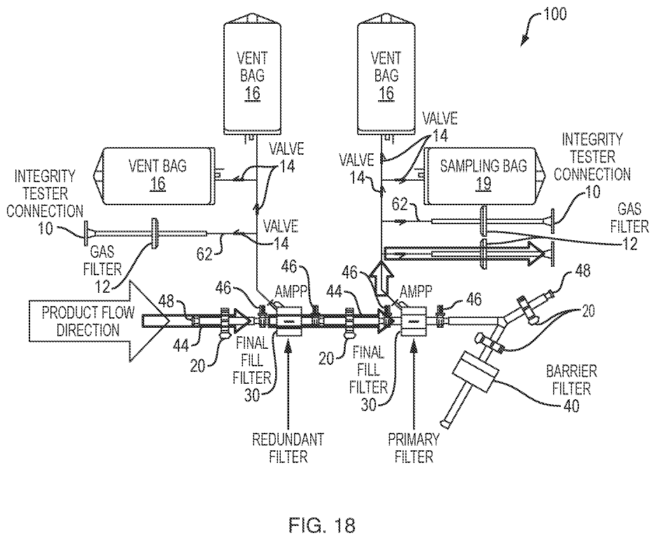

FIG. 18 shows some embodiments of a method of testing the redundant filter whereby the air enters through the inlet of the filter, travels through the filter and exits the vent on the primary filter. FIG. 18 depicts a flow direction of pressurized air during the integrity testing of the redundant final fill filter on some embodiments of a streamlined redundant filtration assembly.

### Integrity Testing of the Redundant Filter on the Traditional Assembly Using Product Inlet for Air-Source and Vent on the Primary Filter as an Outlet

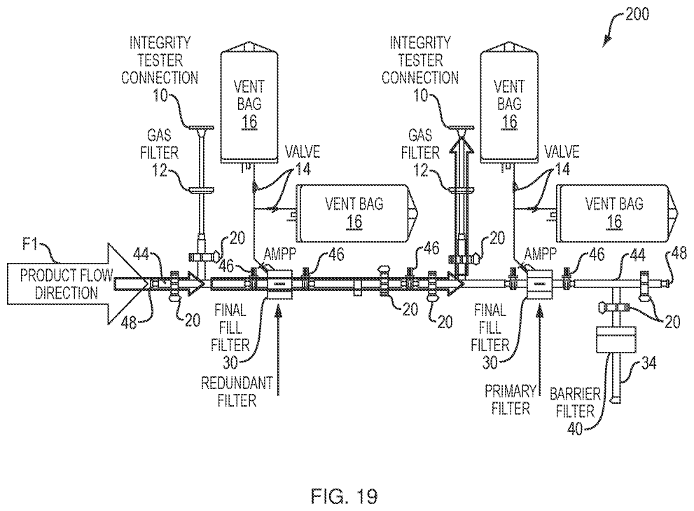

FIG. 19 shows some embodiments of a method of testing the redundant filter whereby the air enters through the inlet of the filter, travels through the filter and exits the assembly through the air inlet of the for the primary filter. FIG. 19 depicts a flow direction of pressurized air during the integrity testing of the redundant final fill filter on some embodiments of a redundant filtration assembly, according to some embodiments of the disclosure.

In some embodiments, each container contains, either partially or completely within its interior, an impeller assembly for mixing, dispersing, homogenizing, and/or circulating one or more liquids, gases and/or solids contained in the container.

All ranges for formulations recited herein include ranges therebetween and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4, or 3.1 or more.

Reference throughout this specification to “one embodiment,” “certain embodiments,” “one or more embodiments,” “some embodiments,” or “an embodiment” indicates that a feature, structure, material, or characteristic described in connection with the embodiment is included in at least one embodiment of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment.

Although some embodiments have been discussed above, other implementations and applications are also within the scope of the following claims. Although the specification describes, with reference to some embodiments, it is to be understood that these embodiments are merely illustrative of the principles and applications of the technologies described within this disclosure. It is therefore to be further understood that numerous modifications may be made to the illustrative embodiments and that other arrangements and patterns may be devised without departing from the spirit and scope of the embodiments according to the disclosure. Furthermore, particular features, structures, materials, or characteristics may be combined in any suitable manner in any one or more of the embodiments.

Publications of patents, patent applications and other non-patent references, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

What is claimed is:

1. A streamlined redundant filtration assembly, comprising:
a main conduit for delivering a biological product, the main conduit further comprises:
a primary final fill filter disposed within the main conduit;
a first connector and a second connector at terminal ends of the main conduit;
a clamp is disposed within the main conduit downstream of the first connector;
a redundant final fill filter is disposed within the main conduit;
an air line in fluid communication with the redundant final fill filter is joined to the main conduit, the air line further comprising an integrity test connection at a distal end;
a vent connected to the air line;
at least one vent bag is in fluid communication with the redundant final fill filter;
two clamps are disposed downstream of the redundant final fill filter, wherein a pinch clamp is disposed between the two clamps;
two vent bags, an air line, and optional clamps and a gas filter are in fluid communication with the primary filter;
a clamp is disposed in the main conduit downstream of the primary filter;
a secondary conduit is joined to the main conduit; the secondary conduit further comprises a barrier filter, the barrier filter and the secondary conduit are joined with the main conduit, a pinch clamp is disposed on the main conduit, wherein the main conduit terminates at the second connector.

2. The redundant filtration assembly of claim 1, wherein the integrity test connection is connected to an air supply.

3. The redundant filtration assembly of claim 1, wherein a gas filter is downstream of the integrity test connection.

4. The redundant filtration assembly of claim 1, wherein there are two vent bags.

5. The redundant filtration assembly of claim 1, further comprising a sampling bag.

6. The redundant filtration assembly of claim 4, further comprising a clamp or a valve is disposed on the air line between the two vent bags.

7. The redundant filtration assembly of claim 1, wherein the vent is an aseptic multi-purpose port (AMPP).

8. The redundant filtration assembly of claim 1, further comprising a peristaltic pump having a conduit connected to the integrity test connection at a first end of the conduit.

9. The redundant filtration assembly of claim 1, further comprising more than one integrity test connection.

10. The redundant filtration assembly of claim 1, wherein a second end of the conduit is connected to a different integrity test connection than the first end of the conduit.

11. The redundant filtration assembly of claim 1, further comprising a recirculation vessel.

12. The redundant filtration assembly of claim 1, further comprising a data acquisition system.

13. The redundant filtration assembly of claim 1, wherein the redundant filtration assembly is single-use.

14. The redundant filtration assembly of claim 1, wherein the redundant filtration system comprises stainless steel.

15. The redundant filtration assembly of claim 13, wherein the redundant filtration system comprises stainless steel and single-use components.

16. A method of integrity testing of at least one final fill filter of the redundant filtration assembly of claim 1, the method comprising:
a. flowing a wetting liquid through the final fill filter;
b. introducing pressurized air into the streamlined redundant filtration assembly through the air line further comprising the integrity testing connection at the distal end;
c. draining the assembly of the wetting liquid;
d. passing the pressurized air through the gas filter on the air inlet and through the vent and the final fill filter before exiting the streamlined redundant filtration assembly through an outlet; and
e. performing at least one test selected from the group consisting of: a bubble point test, a diffusion test, a water flow test, and a pressure hold test.

17. The method of claim 16, wherein the vent is an aseptic multi-purpose port (AMPP) vent port.

18. The method of claim 16, further comprising placing a clamp between the primary filter and the redundant filter, thereby avoiding fluid communication between the downstream side of the redundant filter and the air inlet for the primary filter.

19. The method of claim 16, wherein the draining step is performed using a gravity drain.

20. The method of claim 16, wherein the draining step is performed using a blow-down.

21. The method of claim 16, wherein the final fill filter is the primary final fill filter.

22. The method of claim 16, wherein the final fill filter is the redundant final fill filter.

23. The method of claim 16, further comprising closing the AMPP vent port on the primary filter.

24. The method of claim 23, wherein the barrier filter is the final outlet of the pressurized air.

25. The method of claim 16, further comprising opening the AMPP vent port on the primary filter.

26. The method of claim 16, wherein the AMPP vent port is the final outlet of the pressurized air.

27. The method of claim 16, wherein the pressurized air passes sequentially through an air inlet for the redundant final fill filter and the redundant final fill filter and exits the redundant filtration assembly through the AMPP vent port of the redundant final fill filter.

28. The method of claim 16, wherein the pressurized air passes sequentially through an air inlet of redundant final fill filter into the redundant final fill filter and exits the redundant filtration assembly through AMPP vent port of the primary final fill filter.

29. The method of claim 16, wherein the pressurized air passes sequentially through an air inlet of redundant final fill filter into the redundant final fill filter and exits the redundant filtration assembly through an air inlet of the primary final fill filter.
